(19) United States
(12) Patent Application Publication
(10) Pub. No.: US 2025/0281119 A1
(43) Pub. Date: Sep. 11, 2025

(54) MEDICAL SENSOR, AND SENSOR SUPPORTING MEMBER

(71) Applicant: NIHON KOHDEN CORPORATION, Tokyo (JP)

(72) Inventor: Takahiro ABE, Saitama (JP)

(73) Assignee: NIHON KOHDEN CORPORATION, Tokyo (JP)

(21) Appl. No.: 19/068,531

(22) Filed: Mar. 3, 2025

(30) Foreign Application Priority Data
Mar. 11, 2024 (JP) 2024-037121

(51) Int. Cl. A61B 5/00; A61B 5/145; A61B 90/00

(52) U.S. Cl. CPC A61B 5/68335; A61B 5/14542; A61B 5/684; A61B 90/08; A61B 2090/0807; A61B 2560/0462

(57) ABSTRACT

At least one light emitter is configured to emit light. At least one light detector is configured to output a signal corresponding to an amount of incident light. A supporting member has an adhesive face adapted to be attached to a body of a subject. A sheet is peelably attached to the adhesive face. The supporting member includes a first supporting portion, a second supporting portion and a third supporting portion. The first supporting portion supports and covers the light emitter. The second supporting portion supports and covers the light detector. The third supporting portion is located between the first supporting portion and the second supporting portion. The sheet is formed with a foldable portion extending across a portion facing the third supporting portion and including a portion whose cross-sectional shape is changed so as to increase foldability of the sheet.

### CROSS-REFERENCE TO RELATED APPLICATION

The present application is based on Japanese Patent Application No. 2024-037121 filed on Mar. 11, 2024, the entire contents of which are incorporated herein by reference.

### BACKGROUND

The present disclosure relates to a medical sensor provided with a light emitter that emits light, and a light detector that outputs a signal corresponding to an amount of incident light. The present disclosure also relates to a sensor supporting member for attaching the medical sensor to a body of a subject.

Japanese Patent Publication No. 2020-150996A discloses, as an example of a medical sensor, a sensor adapted to be attached to a fingertip of a subject to calculate a transcutaneous arterial oxygen saturation (SpO2) of the subject. The sensor includes a light emitter and a light detector. The light emitter emits light toward a tissue of the fingertip. The light having passed through the tissue is received by the light detector. The light detector outputs a signal corresponding to intensity of the detected light. The sensor includes a supporting member adapted to be wrapped around the fingertip. The supporting member supports the light emitter and the light detector. The signal that is outputted from the light detector is transmitted through a cable to a device that calculates SpO2.

### SUMMARY

It is demanded to improve the convenience of a medical sensor configured such that a light emitter and a light detector are attached to a subject's body using a supporting member.

An illustrative aspect of the present disclosure may provide a medical sensor, comprising:
at least one light emitter configured to emit light;
at least one light detector configured to output a signal corresponding to an amount of incident light;
a supporting member having an adhesive face adapted to be attached to a body of a subject; and
a sheet peelably attached to the adhesive face,
wherein the supporting member including:
   a first supporting portion supporting and covering the light emitter;
   a second supporting portion supporting and covering the light detector; and
   a third supporting portion located between the first supporting portion and the second supporting portion; and
wherein the sheet is formed with a foldable portion extending across a portion facing the third supporting portion and including a portion whose cross-sectional shape is changed so as to increase foldability of the sheet.

An illustrative aspect of the present disclosure may provide a sensor supporting member adapted to attach, to a body of a subject, a medical sensor provided with at least one light emitter configured to emit light and at least one light detector configured to output a signal corresponding to an amount of incident light, the sensor supporting member comprising:
a first supporting portion supporting and covering the light emitter;
a second supporting portion supporting and covering the light detector;
a third supporting portion located between the first supporting portion and the second supporting portion;
an adhesive face adapted to be attached to the body; and
a sheet peelably attached to the adhesive face, and formed with a foldable portion extending across a portion facing the third supporting portion and including a portion whose cross-sectional shape is changed so as to increase foldability of the sheet.

According to the configuration of each of the above illustrative aspects, as the part of the sheet is peeled from the supporting member, folding of the sheet is promoted at the foldable portion, whereby not only the adhesive face can be caused to be partially exposed, but also it is possible to easily maintain a condition that the part of the sheet as peeled is separated from the adhesive face. As a result, not only attachment of the adhesive face as exposed to the body of the subject can be facilitated, but also it is possible to suppress occurrence of a situation that the adhesive face that is excessively exposed obstructs an initial stage of the attachment operation.

In addition, since a part of the sheet is not separated in accordance with the peeling operation, the part of the sheet as peeled may be used as a tab for peeling the remaining part of the sheet. Since it is not necessary to perform the operation of peeling the sheet from the edge of the supporting member multiple times, the operation of exposing the whole of the adhesive face can be smoothly performed. Furthermore, since the peeled sheet to be discarded is not separated into multiple pieces, the discarding operation can be smoothly performed as well.

Therefore, it is possible to improve the convenience of the medical sensor configured such that the light emitter and the light detector are attached to the body of the subject with the supporting member.

### DESCRIPTION OF THE DRAWINGS

FIG. 1 illustrates an appearance of a front face side of a sensor according to an exemplary embodiment.

FIG. 2 illustrates an appearance of a back face side of the sensor of FIG. 1.

FIG. 3 illustrates an appearance of the sensor under a condition that a sheet of FIG. 2 is peeled off.

FIG. 4 illustrates how to attach the sensor of FIG. 1 to a finger of a subject.

FIG. 5 illustrates how to attach the sensor of FIG. 1 to the finger of the subject.

FIG. 6 illustrates how to attach the sensor of FIG. 1 to the finger of the subject.

FIG. 7 illustrates how to attach the sensor of FIG. 1 to the finger of the subject.

FIG. 8 illustrates how to attach the sensor of FIG. 1 to the finger of the subject.

FIG. 9 illustrates a cross-section of a foldable portion along a line IX of FIG. 2 as viewed from an arrowed direction.

FIG. 10 illustrates another exemplary cross-section of the foldable portion.

FIG. 11 illustrates another exemplary cross-section of the foldable portion.

FIG. 12 illustrates an appearance of a sensor according to another exemplary configuration.

### DESCRIPTION OF EMBODIMENTS

Examples of embodiments will be described below in detail with reference to the accompanying drawings. In each of the drawings, the scale is appropriately changed in order to make each element as illustrated have a recognizable size.

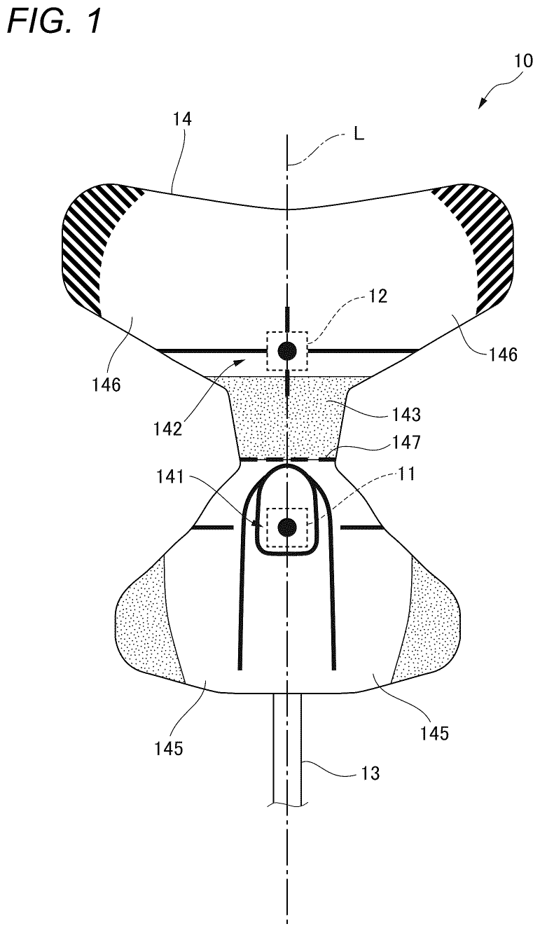

FIG. 1 illustrates an appearance of a sensor 10 according to an exemplary embodiment. The sensor 10 is attached to a fingertip of the subject to calculate a blood light absorber concentration. The sensor 10 is an example of a medical sensor. The fingertip is an example of a body of a subject. Examples of the blood light absorber concentration include a percutaneous arterial oxygen saturation (SpO2).

The sensor 10 includes at least one light emitter 11. The light emitter 11 is a semiconductor light emitter that emits light including a prescribed wavelength. Examples of the semiconductor light emitter include a light emitting diode and a laser diode. The number of light emitter 11 and the value of the wavelength are appropriately determined in accordance with the type of the blood light absorber concentration to be acquired.

The sensor 10 includes at least one light detector 12. The light detector 12 outputs a detection signal corresponding to an amount of light detected on a light detecting face. Examples of the light detector 12 include a photodiode, a phototransistor, and a photoresistor.

The sensor 10 includes a cable 13. The sensor 10 is connected to a pulse photometer (not illustrated) through the cable 13. The cable 13 accommodates a power supply line, a control signal line, and a detection signal line.

The power supply line and the control signal line are connected to a driving circuit (not illustrated) that is connected to the light emitter 11. The power supply line supplies power from the pulse photometer to the drive circuit. The control signal line supplies a control signal transmitted from the pulse photometer to the driving circuit for controlling the activation or deactivation of the light emitter 11.

The power supply line is also connected to the light detector 12. Accordingly, the electric power from the pulse photometer is also supplied to the light detector 12 through the power supply line. The detection signal line electrically connects the light detector 12 and the pulse photometer. Accordingly, the detection signal outputted from the light detector 12 is transmitted to the pulse photometer, and subjected to appropriate processing.

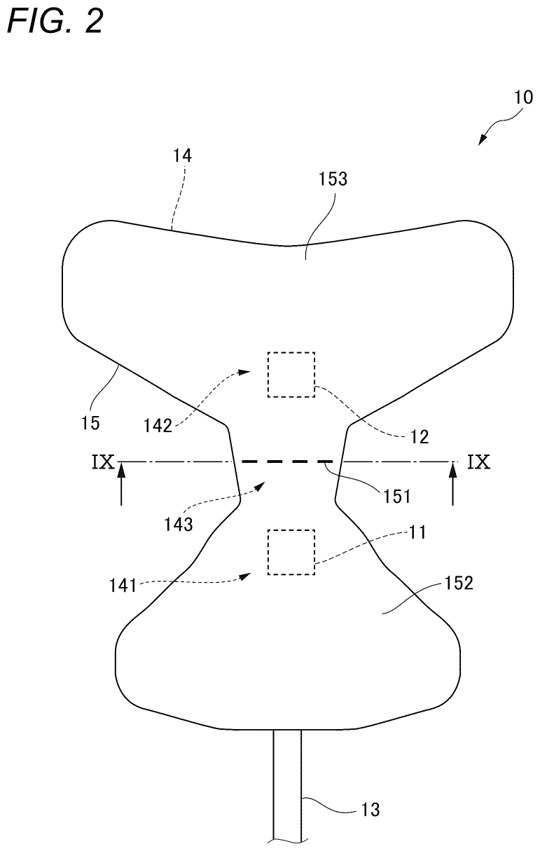

The sensor 10 includes a supporting member 14. FIG. 1 illustrates a front face side of the supporting member 14. FIG. 2 illustrates a back face side of the supporting member 14.

As illustrated in FIG. 1, the supporting member 14 has a first supporting portion 141, a second supporting portion 142, and a third supporting portion 143. The first supporting portion 141 supports and covers the light emitter 11. The second support portion 142 supports and covers the light detector 12. The third supporting portion 143 is located between the first supporting portion 141 and the second supporting portion 142.

As illustrated in FIG. 2, in the initial state, the back face side of the supporting member 14 is covered with a peelable sheet 15. The sheet 15 has a foldable portion 151. The foldable portion 151 extends across a portion of the supporting member 14 facing the third supporting portion 143. As a result, the sheet 15 is sectioned into a first region 152 including a portion facing the first supporting portion 141 of the supporting member 14, and a second region 153 including a portion facing the second supporting portion 142 of the supporting member 14.

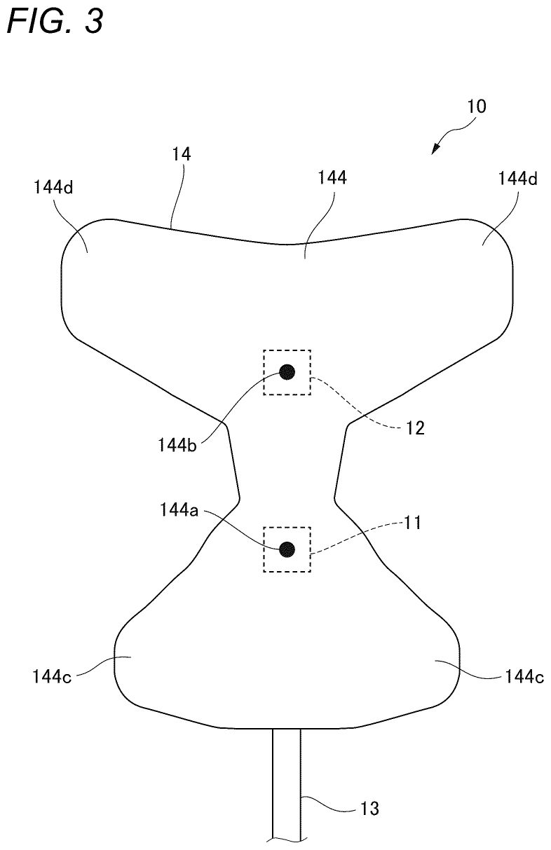

When a user peels the sheet 15 upon the usage of the sensor 10, an adhesive face 144 illustrated in FIG. 3 is exposed. The adhesive face 144 has adhesiveness.

The adhesive face 144 includes a first translucent portion 144a and a second translucent portion 144b. The first translucent portion 144a is disposed so as to face the light emitter 11. The second translucent portion 144b is disposed so as to face the light detector 12.

Each of the first translucent portion 144a and the second translucent portion 144b is formed of a material having transparency with respect to the wavelength of the light emitted from the light emitter 11.

A method of attaching the sensor 10 to a finger 20 of a subject will be described with reference to FIGS. 1 to 8.

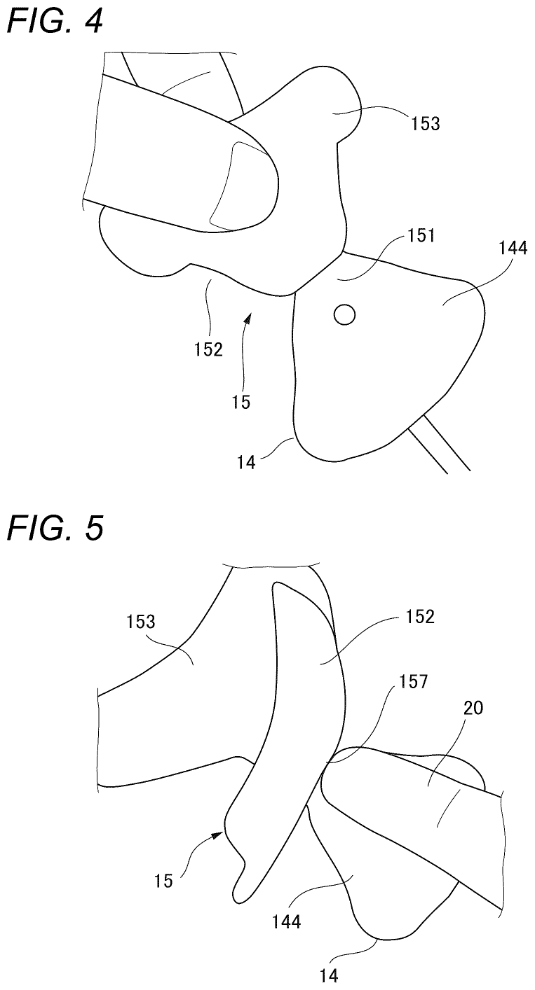

As illustrated in FIG. 4, a user peels the first region 152 of the sheet 15 away from the first supporting portion 141 of the supporting member 14. As a result, a part of the adhesive face 144 located on a back face side of the first supporting portion 141 is exposed.

Subsequently, as illustrated in FIG. 5, a nail side of the finger 20 of the user is placed so as to face the first supporting portion 141. The adhesive face 144 located on the back face side of the first supporting portion 141 is adhered to the nail side of the finger 20. Thereafter, the second region 153 of the sheet 15 is peeled off, so that a part of the adhesive face 144 located on back face sides of the second supporting portion 142 and the third supporting portion 143 is also exposed.

As illustrated in FIG. 1, the supporting member 14 includes a first retaining portion 145. The first retaining portion 145 extends leftward and rightward of the first supporting portion 141. As illustrated in FIG. 3, the adhesive face 144 includes a first adhesive face 144c. The first adhesive face 144c is located on a back face side of the first retaining portion 145.

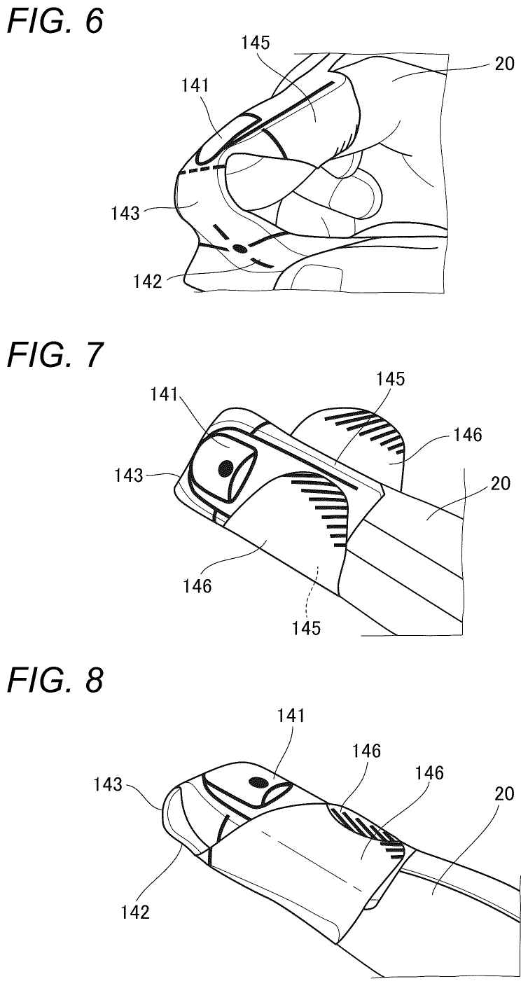

As illustrated in FIG. 6, the first retaining portion 145 is wrapped around the finger 20. As a result, the first adhesive face 144c is adhered to the finger 20. 20

Subsequently, the supporting member 14 is bent while the third supporting portion 143 is faced to a tip of the finger 20, whereby the second supporting portion 142 is disposed so as to face a ball side of the finger 20. The adhesive face 144 located on the back face side of the third supporting portion 143 is adhered to the tip of the finger 20. The adhesive face 144 located on the back face side of the second supporting portion 142 is adhered to the ball side of the finger 20.

As illustrated in FIG. 1, the supporting member 14 includes a second retaining portion 146. The second retaining portion 146 extends leftward and rightward of the second supporting portion 142. As illustrated in FIG. 3, the adhesive face 144 includes a second adhesive face 144d. The second adhesive face 144d is located on a back face side of the second retaining portion 146.

As illustrated in FIGS. 7 and 8, the second retaining portion 146 is wrapped around a portion of the supporting member 14 including the first retaining portion 145. The second adhesive face 144d is adhered to the surface of the first retaining portion 145 while covering the surface. As a result, the retention of the sensor 10 to the finger 20 is finished.

In this state, the light emitter 11 and the light detector 12 are disposed so as to face each other with the finger 20 in between. The light emitted from the light emitter 11 that has passed through the first translucent portion 144a of the supporting member 14 is incident on the tissue of the finger 20. The light that has passed through the tissue and the second translucent portion 144b of the supporting member 14 is incident on the light detecting face of the light detector 12. The pulse oximeter performs processing for calculating a light absorber concentration in arterial blood contained in the tissue based on the detection signal outputted from the light detector 12.

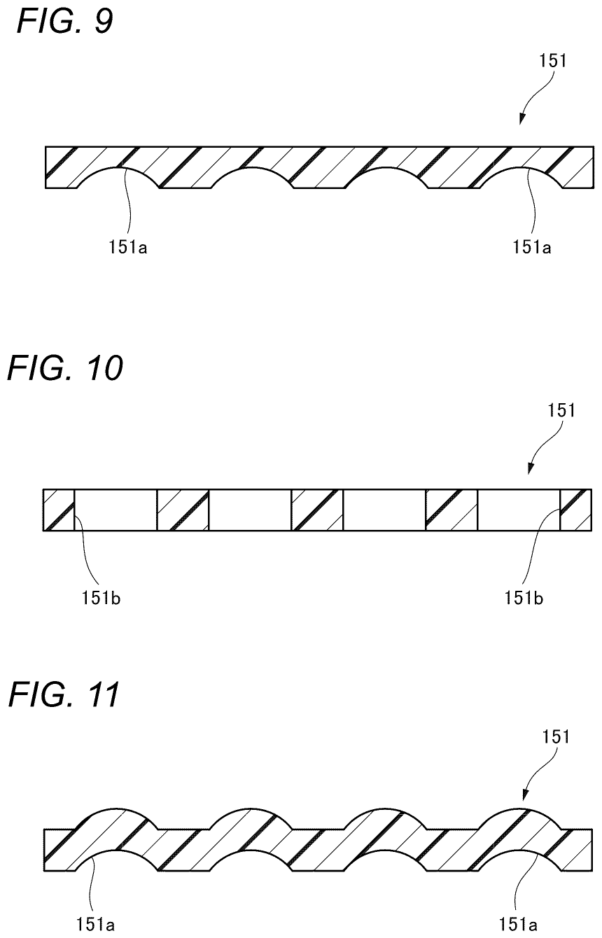

FIG. 9 illustrates a cross-section of the sheet 15 as viewed from the arrowed direction along the line IX illustrated in FIG. 2. The foldable portion 151 includes a portion whose cross-sectional shape is changed so as to increase the foldability of the sheet 15.

Specifically, the foldable portion 151 includes multiple grooves 151a at which the thickness of the sheet 15 is reduced. Each of the multiple grooves 151a is formed by, for example, scoring. As a result, the flexibility of the sheet 15 is enhanced while the first region 152 and the second region 153 are not separated from each other. The number of the groove 151a to be formed may be single.

According to such a configuration, as illustrated in FIG. 4, as the first region 152 of the sheet 15 is peeled from the supporting member 14, folding of the sheet 15 is promoted at the foldable portion 151, whereby not only the adhesive face 144 located on the back face of the first supporting portion 141 can be caused to be partially exposed, but also it is possible to easily maintain a condition that the first region 152 as peeled is separated from the adhesive face 144. As a result, not only attachment of the adhesive face 144 as exposed to the finger 20 can be facilitated, but also it is possible to suppress occurrence of a situation that the adhesive face 144 that is excessively exposed obstructs an initial stage of the attachment operation.

In addition, since a part of the sheet 15 is not separated in accordance with the peeling operation, the first region 152 as peeled may be used as a tab for peeling the second region 153. Since it is not necessary to perform the operation of peeling the sheet 15 from the edge of the supporting member 14 multiple times, the operation of exposing the whole of the adhesive face 144 can be smoothly performed. Furthermore, since the peeled sheet 15 to be discarded is not separated into multiple pieces, the discarding operation can be smoothly performed as well.

Therefore, it is possible to improve the convenience of the sensor 10 configured such that the light emitter 11 and the light detector 12 are attached to the finger 20 with the supporting member 14.

It should be noted that, as illustrated in FIG. 10, the foldable portion 151 may be formed so as to include multiple through-holes 151b. In this case, the foldable portion 151 exhibits a perforated appearance. Even with such a configuration, the foldability of the sheet 15 can be enhanced while the first region 152 and the second region 153 are not separated from each other. The number of through-hole 151b to be formed may be single.

Alternatively, as illustrated in FIG. 11, the multiple grooves 151a may be formed without changing a substantial thickness of the sheet 15. Even with such a configuration, the foldability of the sheet 15 can be enhanced while the first region 152 and the second region 153 are not separated from each other.

As illustrated in FIG. 1, information for assisting attachment of the sensor 10 to the finger 20 is provided on the front face side of the supporting member 14. Such information includes information indicating the position of the light emitter 11, information indicating the position of the light detector 12, information providing a guide indicating the position at which the finger 20 is to be placed, as well as information 147 providing a guide indicating the position at which the supporting member 14 is to be bent when the attachment to the finger 20 is performed (see also FIG. 6). The foldable portion 151 of the sheet 15 is arranged so as to face the information 147 with the adhesive face 144 in between.

According to such a configuration, it is possible to promote association of the operation for folding the sheet 15 at the foldable portion 151 to partially expose the adhesive face 144 with the operation for attaching the supporting member 14 to the finger.

Each of the configurations exemplified above is merely illustrative for facilitating understanding of the present disclosure. Each exemplary configuration may be appropriately modified or combined with another exemplary configuration within the scope of the present disclosure.

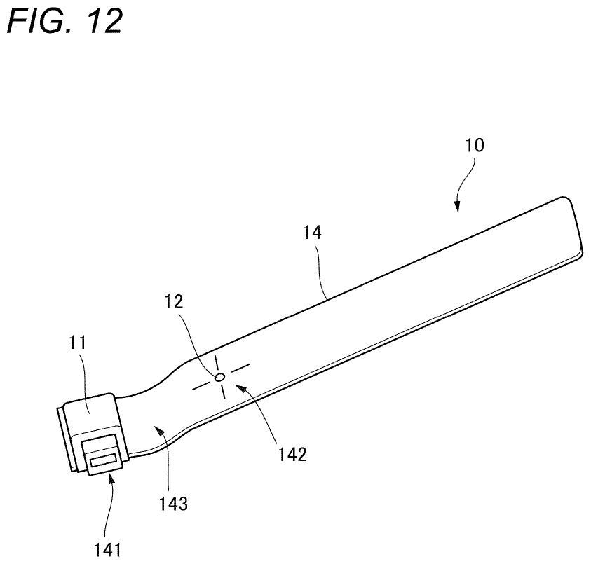

As illustrated in FIG. 1, the supporting member 14 according to the above exemplary embodiment has a shape that is axisymmetric with respect to a straight line L connecting the light emitter 11 and the light detector 12. According to such a configuration, the attachment stability of the sensor with respect to the finger of the subject can be enhanced. However, as illustrated in FIG. 12, the supporting member 14 may exhibit a belt shape

in which the light emitter 11 is supported at one end thereof. In this example, fixation of the sensor 10 is performed by wrapping the supporting member 14 around the finger of the subject. The light detector 12 is supported at a position facing the light emitter 11 with the finger in between when the supporting member 14 is wrapped around the finger.

The application of the medical sensor to which the concept of the present disclosure may be applied is not limited to the detection of the blood light absorber concentration. In the case of a medical sensor that optically detects a physiological parameter, the fundamental concept of the present disclosure can be applied through an appropriate shape change.

1. A medical sensor, comprising:
at least one light emitter configured to emit light;
at least one light detector configured to output a signal corresponding to an amount of incident light;
a supporting member having an adhesive face adapted to be attached to a body of a subject; and
a sheet peelably attached to the adhesive face,
wherein the supporting member including:
a first supporting portion supporting and covering the light emitter;
a second supporting portion supporting and covering the light detector; and
a third supporting portion located between the first supporting portion and the second supporting portion; and
wherein the sheet is formed with a foldable portion extending across a portion facing the third supporting portion and including a portion whose cross-sectional shape is changed so as to increase foldability of the sheet.

2. The medical sensor according to claim 1,
wherein the foldable portion includes a groove at which thickness of the sheet is reduced.

3. The medical sensor according to claim 1,
wherein the foldable portion includes at least one through-hole.

4. The medical sensor according to claim 1,
wherein a surface of the supporting member is provided with information for assisting attachment to the body of the subject; and
wherein the foldable portion is arranged so as to face the information with the adhesive face in between.

5. The medical sensor according to claim 1,
wherein the supporting member exhibits an axisymmetric shape relative to a straight line connecting the light emitter and the light detector.

6. A sensor supporting member adapted to attach, to a body of a subject, a medical sensor provided with at least one light emitter configured to emit light and at least one light detector configured to output a signal corresponding to an amount of incident light, the sensor supporting member comprising:
a first supporting portion supporting and covering the light emitter;
a second supporting portion supporting and covering the light detector;
a third supporting portion located between the first supporting portion and the second supporting portion;
an adhesive face adapted to be attached to the body; and
a sheet peelably attached to the adhesive face, and formed with a foldable portion extending across a portion facing the third supporting portion and including a portion whose cross-sectional shape is changed so as to increase foldability of the sheet.
